(19) United States
(12) Patent Application Publication
(10) Pub. No.: US 2013/0281954 A1
(43) Pub. Date: Oct. 24, 2013

(54) FIT INDICATORS FOR PULL ON TYPE ABSORBENT ARTICLE

(71) Applicant: THE PROCTER & GAMBLE COMPANY, Cincinnati, OH (US)

(72) Inventors: Kaoru ISHIHARA, Cincinnati, OH (US); Tina Marie GLAHN, Cincinnati, OH (US); Koichi MORIMOTO, Beijing (CN)

(73) Assignee: The Procter & Gamble Company, Cincinnati, OH (US)

(21) Appl. No.: 13/868,550

(22) Filed: Apr. 23, 2013

(30) Foreign Application Priority Data
Apr. 23, 2012 (CN) 2012074515

(51) Int. Cl. A61F 13/49

(52) U.S. Cl. CPC A61F 13/49011
USPC 604/385.3

(57) ABSTRACT

Pull-on disposable absorbent articles having a fit indicator are disclosed. Pull-on disposable articles having an extensible belt transversely disposed with respect to a chassis portion are disclosed. The belt portion of the pull-on article is provided with a fit indicator graphic. When the belt portion of the article is unstretched the fit indicator graphic has a first appearance. When the belt portion of the article is stretched to its design range for wear the fit indicator graphic has a second appearance. When the belt portion of the article is stretched beyond its design range for wear the fit indicator graphic has a third appearance. By confirming the configuration of the fit indicator graphic in the second appearance in wear, the proper fit of the disposable absorbent article may be verified. Use of fit indicator graphics in combination with reference graphics and in arrays of products are also disclosed.

### CROSS-REFERENCE TO RELATED APPLICATION

This application claims priority to Chinese Patent Application Number 2012/074515, filed on Apr. 23, 2012, the substance of which is incorporated herein by reference.

### FIELD OF THE INVENTION

The present invention relates to disposable pull-on type garments.

### BACKGROUND OF THE INVENTION

Infants, babies, small children, and other incontinent individuals often wear disposable absorbent articles such as diapers to receive and contain urine, fecal matter, and other bodily exudates. Such disposable absorbent articles come in at least two basic designs. One such design is an “open” or “taped” design in which the product waist is in an open configuration prior to placement about the torso of the wearer. As part of the application process, the product is closed about the wearer's waist such as with taped or mechanical fasteners. Another common design is a “closed” or “pull-on” design characterized by a product with a closed, and typically stretchable, waist configuration. The product is usually expanded about the waist and legs of the wearer for application and contracts (such as with the use of elastic contracting materials) once properly in pace. This second type of product, the so-called “pull-on” or “pant” type is popular for use with children who are toilet training, able to walk, or simply as an alternative to the “open” or “taped” design.

It is understood that proper fit of a pull-on absorbent article is important for fully satisfactory performance of the product. Typically, such pull-on type absorbent articles are held in place on the body of the wearer by elastic contracting members located around the waist opening and the leg openings of the article. A common characteristic of such pull-on absorbent articles is that the waist opening may be expanded over a range which is substantially larger than the optimal waist opening size corresponding to the most desired elastic fit forces. This ability to “overstretch” is often desired to ensure that the pull-on article can be most easily applied even in the face of potential obstructions, such as if the if the wearer is wearing shoes.

Despite the ability of some pull-on type absorbent articles to stretch in the waist more than is necessary during actual wear, it is desirable that the in-use waist stretch be within a more narrow range. This is so that the forces generated by the waist elastic members will be in the optimal range for a given wearer. If forces generated by such elastic contracting members are insufficiently low, the product may sag or slip—particularly as it becomes loaded with urine or other exudates. Contrastingly, if the forces generated by the elastic contracting members are too high, caregivers or wearers may experience difficulty in application or removal of the product. Additionally, if elastic contraction forces are too high the product may create excess pressure on the body of the wearer leading to red marking or other undesired usage experiences. Even if the forces generated by elastic contracting members extended beyond the optimal range are not unsatisfactorily high, other drawbacks may arise. For example, the fact that such a product may physically initially fit on a given wearer—even though the resulting extension of the elastic members is beyond the optimal range—may conceal the fact that such a wearer is likely too large for the product and consequently may load the product with more urine than that particular sized product is designed to handle. This negative product performance could often be avoided if such a wearer was fitted with another sized garment such that the in-use extension of the elastic contracting members was within optimal range for that particular garment design.

While the importance of proper fit is understood by the manufacturers of pull-on type absorbent articles, consumers often experience difficulty in selecting the properly sized product offering for their use. This may be especially true in the case of pull-on absorbent articles designed for babies and small children. It may be even more particularly the case when, as noted above, the physical amount to which the product openings may be stretched encompasses a much broader range than the size at which the product best achieves its design fit. Often products are suggested for appropriate fit by proxy information expected to be readily known by most purchasers. For example, a weight range given on the product package is a typical manner in which selection information is given to consumers for proper product selection. While this approach may work in some circumstances, weight does not necessarily correspond directly to the physical characteristics of the wearer that most impact the ability to get optimal fit from a given design of pull-on absorbent articles. The physical characteristics of the wearer most important for optimal product fit will vary somewhat depending on the specifics of the design pull-on type absorbent article being considered. These characteristics include size, but also relative sizes of body areas, range of motion characteristics, and other attributes of the wearer which may not be well-estimated by weight alone.

A need, therefore, exists for a pull-on disposable absorbent article provided with an improved and easy to use fit assessment feature specifically design to correspond to design of the pull-on absorbent article itself.

### SUMMARY OF THE INVENTION

The present invention is directed to a disposable pull-on absorbent article having a waist opening and a pair of leg openings. The article comprises chassis having a front portion, a back portion and a crotch portion therebetween. The article further comprises a belt portion. The belt portion comprises a front belt portion, a back belt portion, and left and right side belt portions, The belt portion is disposed transversely to said chassis and the side belt portions extend transversely outward from the chassis. The belt portion defines the waist opening. Each of the front belt portion and back belt portion comprises elastomeric material, wherein said waist opening is elastically stretchable and contractable. The elastomeric materials in the belt portion are configured such that the waist opening is characterized by a positive strain design fit range representing less than the full range over which said waist opening may be stretched. The article further comprises a fit indicator graphic disposed in the belt portion thereof. When the waist opening of the article is strained within a range from its fully contacted state up to the design fit range, the fit indicator graphic has a first appearance. When the waist opening of the article is strained within the design fit range, the fit indicator graphic has a second appearance. When the waist opening of the article is strained beyond the design fit range, the fit indicator graphic has a third appearance. By visually confirming that the fit indicator graphic has said second appearance when the article is being worn by a wearer, the correspondence of the wearer to the design fit range of the article may be verified.

In another aspect of the present invention, the article may further comprise a reference graphic disposed on the chassis outside of the belt region. The reference graphic coordinates with said second appearance of said fit indicator graphic. In yet another aspect, this coordination between the reference graphic and the fit indictor graphic is identity of appearance. Alternatively, the coordination between the reference graphic and the fit indicator graphic is complementary appearance.

In another aspect of the present invention, the reference graphic may comprise a reference color and the fit indicator graphic matches the reference color when said fit indictor graphic has its second appearance. In a variation of this aspect, the indicator graphic in the first appearance, the second appearance, and the third appearance vary in color. In another variation, the fit indicator graphic in the first appearance, the second appearance, the said third appearance, may vary in intensity of a substantially similar color.

In another aspect of the present invention, the full range over which the waist opening may be stretched exceeds the design fit range by at least 100%, or in yet another aspect by at least 200%.

In another aspect of the present invention, the invention may comprise an array of disposable pull-on articles. The array comprises a first article according to the present invention and a second article according to the present invention. The design fit range of the first article is smaller than the design fit range of the second article. The range of waist opening strain corresponding to said third appearance of the fit indicator graphic of the first article at least partially overlaps the design fit range of said second article in the array.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1 is a front view of a disposable pull-on absorbent article having a fit indicator graphic according to the present invention.

FIG. 2 is a top plan view of the pull-on absorbent article of FIG. 1 in its flat, uncontracted condition showing the inner surface.

FIG. 3 is a cross section of FIG. 2 taken along line 3-3.

FIG. 4 is a cross section of FIG. 2 taken along line 4-4.

FIG. 5 is a representation of a fit indicator graphic of the present invention in its first (understretched) condition.

FIG. 6 is a representation of the fit indicator graphic of FIG. 5 in its second (design fit stretched) condition.

FIG. 7 is a representation of the fit indicator graphic of FIG. 5 in its third (overstretched) condition.

FIG. 8 is an alternative representation of a fit indicator graphic of the present invention in its first condition.

FIG. 9 is a representation of the fit indicator graphic of FIG. 8 in its second condition.

FIG. 10 is a representation of the fit indicator graphic of FIG. 8 in its third condition.

FIG. 11 is a second alternative representation of a fit indicator graphic of the present invention in its first condition.

FIG. 12 is a representation of the fit indicator graphic of FIG. 11 in its second condition.

FIG. 13 is a representation of the fit indicator graphic of FIG. 11 in its third condition.

FIG. 14 is a reference graphic according to the present invention corresponding to the fit indicator graphic represented in FIGS. 11-13.

### DETAILED DESCRIPTION OF THE INVENTION

As used herein, the terms,” “pull-on absorbent article,” “Pull-on garment, Pant” and “pant-like garment” are intended to be interchangeable and refer to disposable absorbent articles having a pre-formed waist and leg openings. A pull-on absorbent article may be donned by inserting a wearer's legs into the leg openings and sliding the article into position about the wearer's lower torso. These articles may also be commonly referred to as “closed diapers,” “prefastened diapers,” “pull-on diapers,” “training pants,” “diaper-pants,” and “preclosed diapers” all of which terms are intended to have the same meaning as this definition.

The term “disposable” is used herein to describe garments which are not intended to be laundered or otherwise restored or reused as a garment (i.e., they are intended to be discarded after a single use and, preferably, to be recycled, composted or otherwise disposed of in an environmentally compatible manner).

“Absorbent article” refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like. Pull-on Absorbent articles are generally worn by Infants, babies, children and other incontinent individuals to absorb and contain urine, feces or other bodily fluids.

As used herein, the term “joined” encompasses configurations whereby an element is directly secured to another element by affixing the element directly to the other element, and configurations where an element is indirectly secured to another element by affixing the element to intermediate member(s) which are in turn affixed to the other element.

“Proximal” and “Distal” refer respectively to the location of an element relatively near to or far from the longitudinal or lateral centerline of a structure (e.g., the proximal edge of a longitudinally extending element is located nearer to the longitudinal centerline than the distal edge of the same element is located relative to the same longitudinal centerline).

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Longitudinal” refers to a direction running substantially perpendicular from a waist edge to an opposing waist edge of the article and generally parallel to the maximum linear dimension of the article. Directions within 45 degrees of the longitudinal direction are considered to be “longitudinal”

“Tranverse” or “Lateral” refers to a direction running from a longitudinally extending edge to an opposing longitudinally extending edge of the article and generally at a right angle to the longitudinal direction. Directions within 45 degrees of the lateral direction are considered to be “lateral.”

“Disposed” refers to an element being located in a particular place or position.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure, e.g., hydrostatic pressure (aside from natural forces such as gravity). A layer or a layered structure that is water-impermeable according to this definition may be permeable to water vapor, i.e., may be “vapor-permeable.”

“Extendibility” and “extensible” mean that the width or length of the component in a relaxed state can be extended or increased.

“Elasticated” and “elasticized” mean that a component comprises at least a portion made of elastic material.

“Elongatable material,” “extensible material,” or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at no more than 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Elastomeric material” is a material exhibiting elastic properties. Elastomeric materials may include elastomeric films, scrims, nonwovens, and other sheet-like structures.

“Identical” means the objects being compared are the same (e.g., backsheet film A compared to backsheet film B, topsheet A compared to topsheet B, chassis A compared to chassis B, portions of article A compared to the same portions of article B, etc.).

“Substantially identical” means the objects being compared have such close resemblance as to be essentially the same—as understood by one having ordinary skill in the art. “At least substantially identical” encompasses “identical.”

“Stretch” and “Strain” have the same meaning and refer to the elongation of a material when it is subject to a stretching force. An elastomeric material will tend to generate a return force when subject to strain as it attempts to relax toward its initial configuration. As such, there is a relationship between degree of stretch or strain of an elastomeric material and the forces tending toward return such a material generates.

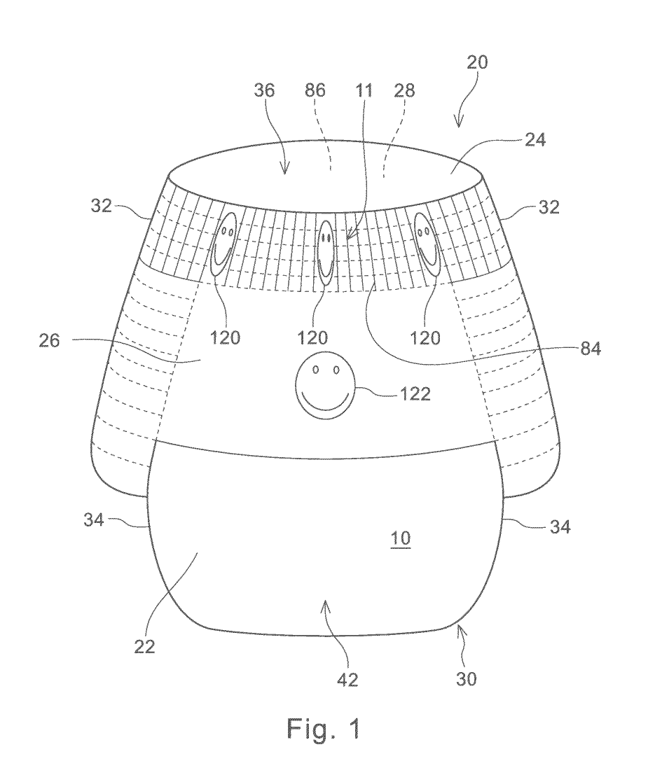

A preferred embodiment of the pull-on garment of the present invention is the disposable absorbent pull-on garment, pull-on diaper 20, shown in FIG. 1. As used herein, the term “pull-on diaper” refers to pull-on garments generally worn by infants and other incontinent individuals to absorb and contain urine and feces. It should be understood, however, that the present invention is also applicable to other pull-on garments such as training pants, incontinent briefs, feminine hygiene garments or panties, and the like. As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.)

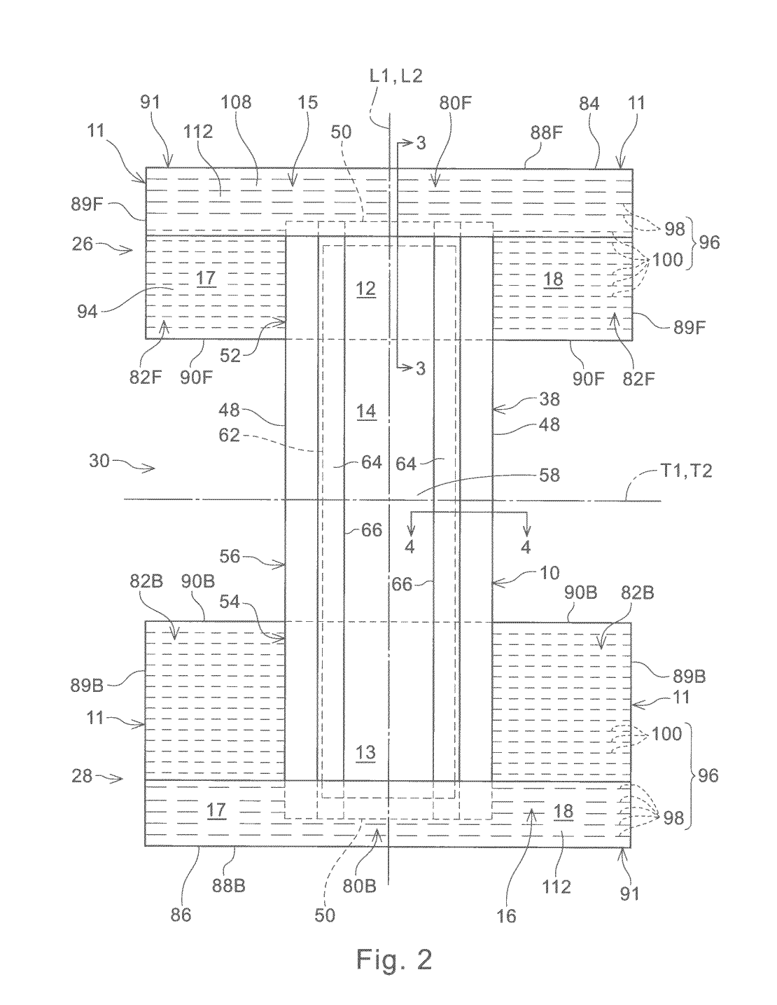

FIG. 1 is a front view of the disposable pull-on absorbent article 20 of the present invention. The pull-on absorbent article 20 has a longitudinal centerline L1 and a transverse centerline T1 (refer to FIG. 2 as well). The pull-on absorbent article 20 has an outer surface 22, an inner surface 24 opposed to the outer surface 22, a front region 26, a back region 28, a crotch region 30, and seams 32 which join the front region 26 and the back region 28 to form two leg openings 34 and a waist opening 36. Also referring to FIG. 2, the pull-on absorbent article 20 comprises a chassis 10 and a belt portion 11. The chassis 10 extends longitudinally along the longitudinal centerline L1 (shown in FIG. 2) from the front region 26 through the crotch region 30 to the back region 28. The chassis 10 comprises a front portion 12, a back portion 13 and a crotch portion 14 therebetween. The belt portion 11 extends transversely outwardly from the chassis 10. The belt portion 11 comprises a front belt portion 15 in the front region 26 and a back belt portion 16 in the back region 28. Each of the front belt portion 15 and back belt portion 16 extends transversely outwardly from the longitudinal centerline L1 to define left belt portions 17 and right belt portions 18.

FIGS. 1-3, show one example of a disposable pull-on absorbent article 20 according to the present invention. As shown in FIGS. 1 and 2, the pull-on absorbent article 20 comprises an absorbent chassis 10 to cover the crotch region of the wearer and a belt portion 11 extending transversely about the waist opening 36. The pull-on absorbent article 20 may also comprise an outer cover layer 42 to cover the chassis 10. The belt portion 11 defines the waist opening 36. The belt portion 11 and the chassis 10 and/or the outer cover layer 42 jointly define the leg opening 34. Alternatively, the belt portion 11 and the outer cover layer 42 may jointly define the leg opening 34.

The absorbent main body 38 absorbs and contains body exudates deposited on the chassis 10, most commonly in the crotch portion 14 of the main body In the embodiment shown in FIG. 2, the main body 38 has a generally rectangular shape having a longitudinal centerline L2, a transverse centerline T2, left and right longitudinally extending side edges 48 (hereinafter may be referred to as “longitudinal side edge”) and front and back transversely extending end edges 50 (hereinafter may be referred to as “transverse end edge”). The main body 38 also has waist panels (i.e., a front waist panel 52 positioned in the front waist region 26 of the pull-on article 20 and a back waist panel 54 positioned in the back waist region 28) and a crotch panel 56 in the crotch region 30 between the front and back waist panels 52, 54.

The belt portion 11 comprises a front belt portion 15 and a back belt portion 16 (hereinafter may be referred to as “front and back belt 15, 16) and has a ring-like configuration by connecting the front belt 15 and the back belt 16. Each of the front belt 15 and the back belt 16 has a central panel 80F, 80B and side panels 82F, 82B contiguous with the central panel 80F, 80B and extending transversely outwardly from the central panel 80F, 80B. Thus, the belt portion 11 comprises a central panel 80F, 80B and the side panels 82F, 82B. Herein, a portion of a front member and a portion of a back member may be indicated by a reference number with “F” suffix and “B” suffix, respectively, as necessary. Therefore, the “central panel 80F, 80B” for example indicates the “front central panel 80F” and the “back central panel 80B”. The “central panel 80” also may mean the “front central panel 80F” and the “back central panel 80B”.

Each of the front belt 15 and the back belt 16 has a transversely extending distal edge 88F, 88B, a transversely extending proximal edge 90F, 90B, and longitudinally extending left and right side edges 89F, 89B. Herein, the term “proximal” is used to indicate the position of a “proximal” portion being closer relative to the crotch panel of the chassis than the position of a “distal” portion. Therefore, the proximal edge 90F, 90B is located closer than the distal edge 88F, 88B relative to the crotch panel 56 of main body 38. The front and back belts 15, 16 are joined at or adjacent to the side edges 89F, 89B at the seams 32 to form a pull-on article having a waist opening 36 and two leg openings 34. The front central panel 80F may partly or entirely overlap with the front waist panel 52 of the chassis 10. The back central panel 80B may partly or entirely overlap with the back waist panel 54 of the chassis 10. However, the central panel 80F, 80B does not extend into the crotch portion 14 of the chassis 10 and is not disposed in the crotch portion 14. In the embodiment shown in FIG. 2, the central panel 80F, 80B partly overlaps with and is joined to the front waist panel 52 and the back waist panel 54, respectively.

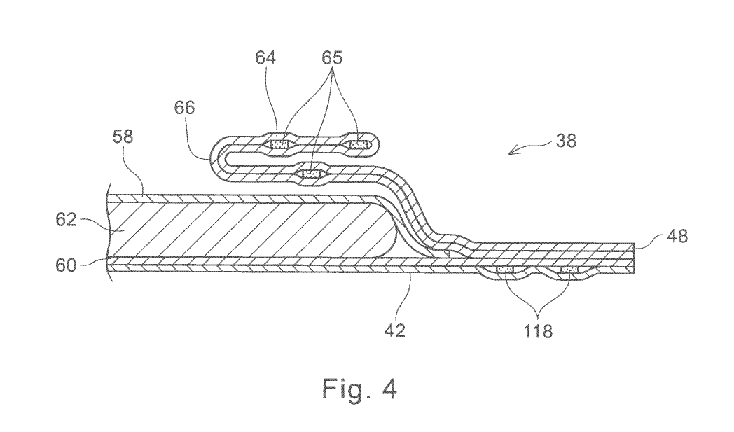

The chassis 10 and the belt portion 11 may be formed with separate elements. For example, the belt portion 11 may be joined by any known means to the chassis 10. In the embodiment shown in FIGS. 1, 2, and 3 the chassis 10 comprises the waist panels 52, 54 of the main body 38, and the crotch panel 56 of the main body 38. The main body 38 comprises a liquid pervious topsheet 58, a liquid impervious backsheet 60 and an absorbent core 62 disposed therebetween. The main body 38 may additionally comprise a barrier leg cuff 64 disposed along the longitudinal side edge 48. The barrier leg cuff 64 provides improved containment of liquids and other body exudates in the crotch region 30. The barrier leg cuff 64 may have any of the features or designs known in the art for pull-on absorbent articles. For example, the barrier leg cuff 64 may commonly be provided with elastic material in one or several strands such as leg cuff strands 65 (FIG. 4). The barrier leg cuffs 64 may be folded in a variety of configurations relative to such elastics in order to get the “stand up”, leg gasketing or other desired characteristics.

The liquid pervious topsheet 58 is preferably positioned adjacently the body-facing surface of the absorbent core 62 and may be joined thereto and/or to the backsheet 60 by any attachment means known in the art. The liquid impervious backsheet 60 is generally that portion of the pull-on absorbent article 20 positioned adjacently the garment-facing surface of the absorbent core 62 and prevents the exudates absorbed and contained therein from soiling articles that may contact the pull-on absorbent article 20. The absorbent core 62 is positioned between the topsheet 58 and the backsheet 60 and absorbs and retains liquids such as urine and other certain body exudates. The topsheet 58, the backsheet 60 and the absorbent core may be manufactured any known materials. Suitable topsheet materials may include porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Suitable backsheet materials may include breathable materials that permit vapors to escape from the diaper while still preventing exudates from passing through the backsheet. Suitable absorbent core materials may include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

In the example of the invention shown in FIGS. 1-3, the outer cover layer 42 is disposed on the outer surface 22 of the pull-on absorbent article 20 and covers the crotch panel 56 of the absorbent main body 38. The outer cover layer 42 may extend into and cover the front waist panel 52 and the back waist panel 54 of the main body 38. The outer cover layer 42 is directly joined to and covers the liquid impervious backsheet 60 of the main body 38. The central panel 80 of the front and back belt 15, 16 portion is joined to the front waist panel 52 and the back waist panel 54 of the main body 38 through the outer cover layer 42.

Thus, as shown in this example, the outer cover layer 42 may be sandwiched between the front and back belt 15, 16 and the liquid impervious backsheet 60 of the main body 38. The outer cover layer 42 may be, but need not be, coextensive with the liquid impervious backsheet 60. As shown in FIG. 4, leg elastic material 118 may be disposed so as to extend generally longitudinally along the longitudinal side edge 48 of the main body 38. The leg elastic material 118 may be disposed at least in the crotch region 30 of the pull-on article 20 or may be disposed along the entirety of the longitudinal side edge 48.

The outer cover layer 42 may comprise a material separate from the material of the inner layer 94 and the outer layer 92 constituting the belt 11. The outer cover layer 42 may comprise two of more layers of material. The outer cover layer 42 may comprise any known materials and may comprise materials as used for the front and back belt 15, 16 as explained above. Preferably the outer cover layer 42 comprises a single layer of nonwoven web of synthetic fibers.

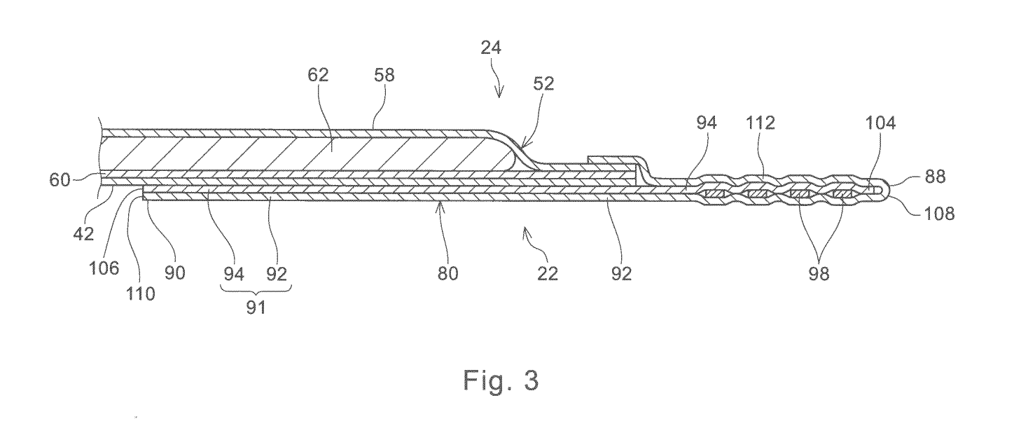

The belt portion 11 may be ring-like and elastic. The ring-like elastic belt portion 11 extends transversely about the waist opening 36 of the pull-on article 20 and acts to dynamically create fitment forces and to distribute the forces dynamically generated during wear. The ring-like elastic belt portion 11 may comprise a belt layer 91 (as shown in FIG. 3) including an outer layer 92. The belt layer 91 may further comprise an inner layer 94. A belt elastic material 96 is interposed between the outer layer 92 and the inner layer 94. The front belt portion 15 and the back belt portion 16 may comprise the same materials and/or may have the same structure. Alternatively, the front belt portion 15 and the back belt portion 16 may comprise different materials and/or may have different structures. In the embodiment of FIG. 2, the front belt portion 15 and the back belt portion 16 generally have the same layer structure. Referring to FIG. 3, the inner layer 94 has a transversely extending distal end 104 and a transversely extending proximal end 106. The outer layer 92 has a transversely extending distal end edge 108 and a transversely extending proximal end edge 110. The inner layer 94 is almost coextensive with the contour of the front and back belt portions 15, 16. Alternatively, the inner layer 94 may be smaller than the size of the front and back belt portions 15, 16. The outer layer 92 of the belt layer 91 is longer than the size of the inner layer 94 in the longitudinal direction and an end flap 112 of the outer layer 92 is folded to cover the distal end 104 of the inner layer 94 at the waist opening 36 and to form a distal end edge 108 of the outer layer 92. The inner layer 94 of the belt layer 91 may also have an end flap which may be folded together with the end flap 112 of the outer layer 92. The end flap of the inner layer 94 may be longer or shorter than or equal to the end flap of the outer layer 92. Alternatively, the end flap 112 may be eliminated such that the outer layer 92 terminates at the waist opening 36 to form the distal end edge 108. In the embodiment shown in FIGS. 2 and 3, the distal end edge 108 and the proximal end edge 110 of the outer layer 92 correspond to the distal edge 88 and the proximal edge 90 of the front and back belt 15, 16, respectively. The outer layer 92 surrounded by the distal end edge 108 and the proximal end edge 110 defines the shape of the front and back belt 15, 16 in the embodiment shown in FIGS. 2 and 3.

The front and back belt portions 15, 16 may comprise any known materials. Suitable material for the front and back belt portions 15, 16 can be manufactured from a wide range of materials such as plastic films; apertured plastic films; woven or nonwoven webs of natural materials (e.g., wood or cotton fibers), synthetic fibers (e.g., polyolefins, polyamides, polyester, polyethylene, or polypropylene fibers), or a combination of natural and/or synthetic fibers; or coated woven or nonwoven webs. Preferably the belt comprises a nonwoven web of synthetic fibers. The belt may comprise a stretchable nonwoven. More preferably, the belt comprises an inner hydrophobic, non-stretchable nonwoven material and an outer hydrophobic, non-stretchable nonwoven material.

The belt elastic material 96 comprises a waist elastic material 98 and a side elastic material 100. The waist elastic material 98 may comprise one or more of elastic elements such as strands or panels extending in the transverse direction. The side elastic material 100 also may comprise one or more of elastic elements such as strands or panels extending in the transverse direction. The waist elastic material 98 may be continuously disposed along the distal edge 88 of the front and back belt portions 15, 16. The side elastic material 100 may be disposed in the side panel 82 of the front and back belt portions 15, 16. In the embodiment shown in FIG. 2, the waist elastic material 98 and the side elastic material 100 comprise a plurality of elastic strands which are disposed at a constant interval in the longitudinal direction. Alternatively, the waist elastic material 98 and the side elastic material 100 may be disposed at a different interval in the longitudinal direction. No elastic material may be provided in a portion of the central panel 80 of the front and back belt 15, 16 which overlaps with the absorbent core 62, preferably with the front and back waist panel 52, 54 of the main body 38. Alternatively, no elastic material may be provided in the entirety of the central panel 80. The non-elastic region on the central panel 80 may be formed by cutting the continuously disposed waist elastic material 98 and/or side elastic material 100 in the region of the central panel 80. However, an elastic material may be provided in the central panel 80 if it is necessary.

Typically, the belt elastic material 96 in a stretched condition is interposed and joined between the uncontracted outer layer 92 and the uncontracted inner layer 94. When the belt elastic material 96 is relaxed, the belt elastic material returns to the unstretched condition and contracts the outer layer 92 and the inner layer 94. The belt elastic material 96 may provide a desired variation of contraction force in the area of the ring-like elastic belt 11. For example, the contraction force of the front belt portion 15 may be greater or lower than that of the back belt portion 16. The contraction force of the waist elastic material 98 may be greater than that of the side elastic material 100, which is typically preferable to provide the pull-on article 20 with an anchoring force against the wearer at the waist opening 36.

Pull-on disposable absorbent articles of the present invention are also provided with at least one fit indicator graphic disposed on the belt portion of the article. In the example of the invention shown in FIG. 1, the pull-on disposable absorbent article 20 is shown with a series of fit indicator graphics 120 disposed on the belt portion 11 of the article 20. A least one fit indicator graphic 120 may be disposed in the central panel 80 of the belt portion 11 and more particularly in the front central panel 80F. If more than one fit indicator graphic 120 is used, they may all be disposed in the central panel 80 of the belt portion 11 or they may disposed in a combination of locations in the belt portion 11 such as within the central panel 80 and within the left and right belt portions 17 and 18. One or more fit indicator graphics 120 may also be disposed only within the right or left belt portions 17 and 18 without any being in the central panel 80 of the belt portion.

It is generally desirable that at least one fit indicator graphic 120 be disposed in a location in the belt portion 11 where it will be readily perceivable by the wearer and/or a caregiver. By disposing at least one fit indicator graphic 120 in the front belt portion 15 the fit indicator graphic may be disposed so as to be readily perceivable by both the wearer of the article 20 and by a caregiver. Fit indicator graphics 120 may be disposed in the back belt portion 16 either in addition to the front belt portion 15 or as an alternative to the front belt portion 15—although such an alternative disposition might would be expected to impact the ability of the wearer of the article 20 to observe the fit indicator graphic 120 while the article 20 is in use.

Regardless of the disposition of several fit indictor graphics within the belt portion 11, when multiple fit indictor graphics are used on the same article 20, it is desirable that they have a similar or identical appearance. As will be further discussed the amount of belt portion stretching or strain in a particular location may not be uniform across the belt portion 11 for a given amount of strain applied to the belt portion 11 as a whole. Nevertheless, each fit indicator graphic 120 should be configured such that it transitions from its first appearance to its second appearance and to its third appearance (discussed further below) at a similar in-use strain of the overall belt portion 11 of the article 20—not necessarily the same localized stretching. In other words if the overall stretching forces (such as those applied by the wearer's body to the belt portion 11 in use applied to the article 20 are such that the belt portion 11 is being strained to its design fit range, then all fit indicator graphics 120 applied to the belt portion 11 should transition to a visually similar second appearance. This should occur even if the localized strain within say the central panel of the belt portion 80 differs from the localized strain of say the left belt portion 17.

When no stretching forces are applied to the belt portion 11 of the pull-on article 20, the belt portion 11 is in its fully contracted or “zero-strain” condition. When the pull-on absorbent article 20 is worn by a wearer it is best designed to fit, the belt portion 11 of the article 20 will be said to be stretched to a point within the design fit range. The design fit range corresponds to a waist size range for the intended wearer. When the article is properly in place on a wearer having a waist size within the design fit range, the belt portion 11 of the article 20 will be strained to a point within the design fit range. The elastomeric material 96 within the belt portion 11 will tend to generate return forces which act to anchor the product onto the body of the wearer. It is desirable to balance these anchoring forces with forces which will tend to cause the article 20 to sag or droop such as the forces tending to pull the article downward as it is loaded.

In order for the article 20 to be applied to the body of the wearer, it is typically necessary to expand the waist opening 36 beyond the point which it will be extended in use (i.e. beyond the design fit range). Typically, the belt portion 11 of the article 20 may be stretched significantly beyond the design fit range. For example, the belt portion 11 may be stretchable to amount at least 100% or even 200% more than the strain associated with the middle of the design fit range. While such overstretching is helpful in application of the product (such as to more easily get the waist opening 36 over shoes or the like), it is not desirable to have the belt portion 11 overstretched in use. The term “overstretched” in this context means that the belt portion 11 in use is strained to a total length which exceeds the design fit range of the article. This situation most commonly occurs when the wearer is larger (for example in waist size) than the size of the wearer for whom the product is most optimally designed.

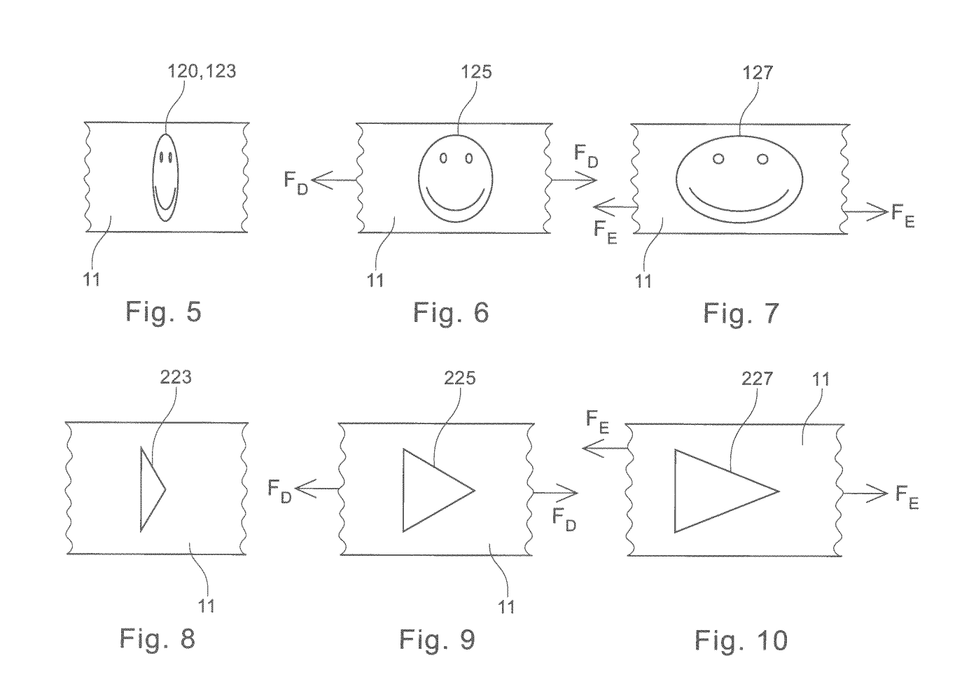

When belt portion 11 of the pull-on article 20 is in its zero-strain condition, the fit indicator graphic will exhibit a first appearance (such as first appearance 123 shown in FIG. 5). Such a first appearance may be indicated by comparison of the fit indicator graphic 120 to a reference graphic 122 such as shown in FIG. 1. The reference graphic 122 is disposed on the chassis 11 of the article 20 outside of the belt region 11. The reference graphic is that of the fit indicator graphic 120, but the reference graphic 122 is shown having the second appearance of the fit indicator graphic (discussed further below). When the article 20 is worn, the reference graphic 122 generally does not undergo a perceptible change in appearance and thus serves as a benchmark or reference for the appearance changes of the fit indicator graphic 120.

It is not necessary to have a reference graphic 122 in all executions of the present invention. The fit indicator graphic 120 may be selected and configured to be “self-referencing.” For example, as shown in FIG. 1, the fit indicator graphic may be a “smiley-face.” In the zero-strain or first appearance of this smiley-face, it is seen as a compressed oval and is no circular or symmetrical. This lack of symmetry provides the clear indication that the fit indicator graphic 120 is in its first configuration corresponding to understretching (compared to the design fit range) of the belt portion 11. Other examples of fit indicator graphics which would be recognized as having or not having longitudinal and transverse symmetry such as circles, triangles, or squares may also be used without the use of a reference graphic.

The fit indicator graphic 120 will be visible in its first appearance while the belt portion 11 is strained within the range from zero-strain (i.e. unstretched) to a point below the design fit range of the article 20. When the belt portion 11 of the article 20 is stretched to a point within the design fit range for the article 20, the fit indicator graphic will be visible in its second appearance. Examples of fit indicator graphics shown in their second appearance are shown in FIGS. 6 and 9 as second appearance fit indictor graphics 125 and 225. In contrast to the first appearance fit indicator graphic 122 shown in FIG. 5, the second appearance fit indictor graphic 125 shown in FIG. 6 is a circle (i.e. is symmetric) and not a compressed oval. Similarly, second appearance fit indicator graphic 225 shown in FIG. 9 is a symmetric (or equilateral triangle) and not a foreshortened triangle such as that shown in FIG. 8 as first appearance indicator graphic 223. As shown in FIGS. 6 and 9, the belt portion 11 is being subjected to stretching forces F_{D} which correspond to the design fit range stretching forces for the pull-on absorbent article (in contrast to FIGS. 5 and 8 where no external forces are being applied).

In situations in which a reference graphic 122 is used in present invention, the reference graphic 122 may have the same visible appearance as the fit indicator graphic in its second appearance. This allows the user to compare the fit indicator graphic to the reference graphic 122 to check for similarity. When the graphics appear substantially identical, the user can verify that the product is being worn within its design fit range.

When the belt portion 11 of the article 20 is stretched beyond its design fit range, the fit indicator graphic 120 will be visible in its third appearance. Examples of fit indicator graphics shown in their third appearance are shown in FIGS. 7 and 10 as third appearance fit indictor graphics 127 and 227. In contrast to the second appearance fit indicator graphic 125 shown in FIG. 6, the third appearance fit indictor graphic 127 shown in FIG. 7 is an elongated oval and not a symmetric circle. Similarly, third appearance fit indicator graphic 227 shown in FIG. 10 is an elongated triangle and not a symmetric or equilateral triangle such as that shown in FIG. 9 as second appearance indicator graphic 225. As shown in FIGS. 7 and 10 the belt portion 11 is being subjected to stretching forces F_{E} corresponding to magnitude which exceeds the design fit range stretching forces for the pull-on absorbent article.

In situations in which a reference graphic 122 is used in present invention, the fit indicator graphic 120 in its third appearance will appear transversely elongated in comparison to the reference graphic. This allows the user to compare the fit indicator graphic to the reference graphic 122 to confirm that the product is being worn in a stretch configuration exceeding the design fit range of the article 20. While it typically will be possible to use a pull-on article with strain of the belt portion 11 exceeding the design fit range, this may not lead to the most desired performance of the product. Therefore, it is desired that the transition of the fit indicator graphic to its third appearance in use be taken as a signal that the user should employ a differently sized pull-on article (e.g. a larger size) which has a design fit range corresponding to the particular wearer.

The fit indicator graphics 120 of the present invention may be provided to the pull-on article 20 in a variety of suitable ways. For example, the fit indicator graphic 120 may be printed directly onto one of the layers of the belt portion 11. One such location may be the outer side of the outer layer 92. Such a location, while resulting in the clearest image, may lead to ink rub off such as onto clothing, carpet or the like depending on the relative adhesion between the ink comprising the graphic and the material comprising the outer layer 92 of the belt portion 11. As such, the fit indicator graphic 120 may also be printed onto the inner surface of the outer layer 92 or onto the outer surface of the inner layer 94. Alternatively, the fit graphic 120 may be printed onto a separate layer, sheet or film (not shown) which may be interposed between the outer layer 92 and inner layer 94 together with the belt elastic material 96. If such a separate sheet, film, or layer is used, it should be disposed so as not to interfere with the stretch and recovery of the belt portion elastic material 96.

In the example shown in FIGS. 1 and 3, the fit indicator graphics 120 are printed onto the outer surface of the inner layer 94. The images comprising the fit indicator graphics 120 may be added to the article 20 by any suitable method of image making. For example, the images may be formed using flexographic printing, ink-jet printing, screen printing, or rotogravure printing. The fit indicator graphics 120 may be printed while the inner layer 94 is in its elongated state (such as before application of the elastic material 96 or after the inner layer 94 has been allowed to contract and gather by operation of the relaxing of the elastic material 96. In either case, the initial configuration in which the fit indicator graphics 120 are printed should be adjusted for any subsequent stretching and gathering such that the relationship between the in use strain of the belt portion 11 and the first appearance 123 the second appearance 125 and the third appearance 127 of the fit indicator graphics 120 is maintained.

To demonstrate the maintaining the relationship between the strain condition of the belt portion 11 and the appearance condition (first, second or third) of the fit indicator graphic, some potential examples will be discussed. In one example, the fit indicator graphics may be applied to the outer surface of the inner layer prior to application of the elastic material (that is, while the inner layer is in its flat condition). In this example, the fit indicator graphics will typically be printed in an elongated or extended configuration (such as that of the third appearance) such that when the inner layer contracts upon construction, the fit indicator graphic will also contract to its first, or foreshortened appearance). The exact shape of the fit indicator graphic should be determined based upon the ultimately desired design fit range of the article such that the fit indicator graphic will appear in the second appearance state when the belt portion of the article is stretched to the design fit range. Similarly, the fit indicator graphic should also be seen in its third appearance state when the belt portion is strained beyond such design fit range.

As another example, the fit indicator graphics 120 may be applied to the belt portion 11 after the elastic material 96 has been allowed to contract. In such a situation, the fit indicator graphics 120 may be applied to an exposed outer surface, for example. Typically, the ridges of the gathered belt portion 11 will be available to receive the image component, but the valleys between the ridges (in the gathered belt portion 11) will not usually receive the image. Nevertheless, as the belt portion 11 is strained the fit indicator graphic 120 will expand with the belt portion 11. The fit indicator graphic 120 in this example is typically applied in the first appearance state such as first appearance 123. The precise initial shape of the fit indicator graphics 120 is chosen such that when the belt portion 11 is expanded to the design fit range of the article 20, the fit indicator graphics 120 will appear in their second state such as second state 125. It will be recognized that during such elongation of the belt portion 11, the fit indicator graphic 120 may appear to develop “gaps”, but the graphic should still be recognizable as a continuous design allowing the user to determine if its overall appearance is within the first appearance state or the second appearance state or the third appearance state. As the article 20 is stretched beyond its design fit range, the fit indicator graphics will expand even further to the third appearance state, such as third appearance state 127.

If a reference graphic such as reference graphic 122 is used, it may also be applied in any suitable manner such as with any of the methods suitable for application of the fit indicator graphic. The reference graphic may also be applied onto one of layers of the article 20 or may be applied on its own layer or material employed for this purpose. For example, a reference graphic 122 may be printed directly onto the outer or inner surface of the outer cover 42 in the chassis portion 10 or may be printed on an additional layer (not shown) disposed inside said outer cover 42.

Another manner of applying the fit indicator graphics of the present invention is to use contrasting solid colors among layers of the belt portion 11. As one example, the belt portion 11 may comprise tinting or other coloration on an outer layer comprising the belt portion. As the belt portion is stretched 11 the appearance of the coloration will appear to change, such as by fading as the color material stretches out. As another example the belt portion may comprise a see-through or slit outer layer with an underlying contrasting color layer. As the belt portion is extended, the outer layer will allow greater visibility through it (either naturally or by slits opening more) revealing more of the color of the underlying contrasting layer. When such color change mechanisms are used for the fit indicator graphic, it may be preferable to use a reference graphic showing an example of the second appearance state of the fit indicator graphic. This is because such color changes are not necessarily naturally perceived as symmetric or asymmetric and, therefore, may not be as readily “self-referencing” as other executions. As another alternative, a reference may be placed elsewhere than on the product itself such as on the package in which the product is provided.

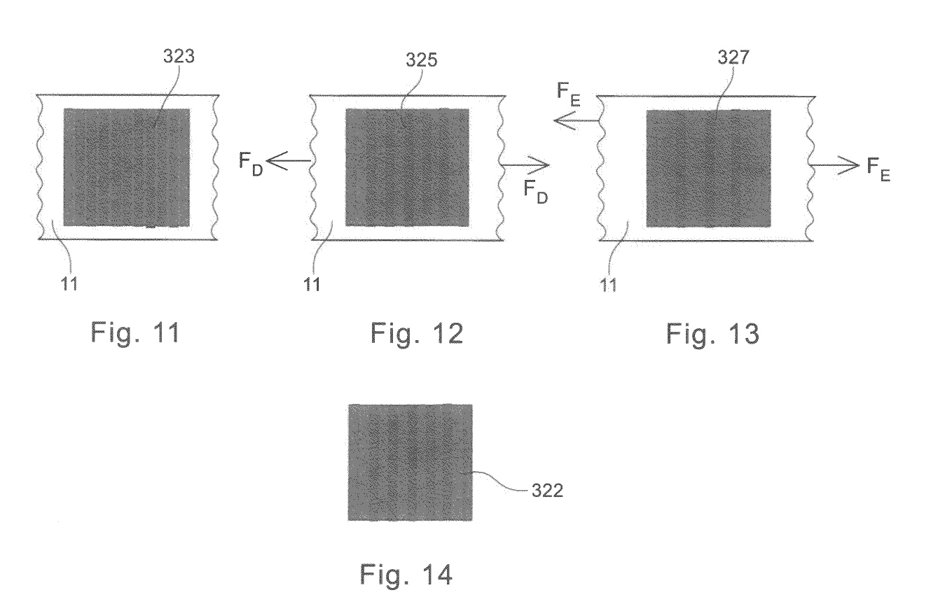

FIGS. 11-14 show one possible example of such a color changing mechanism for the fit indicator graphic. FIG. 11 shows a fit indicator graphic disposed on the belt portion 11 in a first appearance state 324. No stretching load is applied to the belt portion 11. FIG. 12 shows the belt portion 11 subject to its design force loading F_{D} and a fit indictor graphic in a second appearance state 325. FIG. 13 shows the belt portion 11 subject to a stretching force F_{E} exceeding its design force and a fit indicator graphic in a third appearance. FIG. 14 shows a sample of a reference graphic 322 not placed on the article, but located on the chassis portion 10. In the version shown in FIG. 14 the reference graphic 322 coordinates with the fit indicator graphic having the second appearance 325 by being a matching color configuration.

The state of the fit indicator graphic of the present invention may also be determined by its complementing the appearance of a provided reference graphic. As an example (not shown), the reference graphic may comprise part of an image or a scene. The fit indicator graphic may be configured in such a manner that in its second appearance state it best completes or otherwise compliments the appearance of the reference graphic.

Regardless of how the fit indicator graphic is applied to the pull-on article 20 or configured, the relationship between the extent of strain of the belt portion 11 relative to the design fit range and the appearance of the fit indicator graphic(s) in a first appearance, a second appearance, or a third appearance as described above should be maintained. In one example, a pull-on article of the present invention may be classified as a “Medium” or “M” sized product. Such a product may have a total circumference of the waist opening 36 as defined by the belt portion 11 of less than 300 mm when the product is in its relaxed or zero-strain state. In such state, the fit indicator graphic 120 will demonstrate its first appearance such as first appearance 123 or 223. As shown in FIG. 5 and FIG. 8 the belt portion 11 may have no external load or force, or a minimal force applied to it.

The design fit range for such an “M” sized pull-on article may correspond to a waist opening 36 circumference of 420 mm to 450 mm. When the belt portion of such an “M” sized pull-on article is worn by a wearer such that the resulting waist opening circumference is within this 420 mm to 450 mm range the fit indicator graphic 120 should demonstrate its second appearance such as second appearance 125 or 225. FIGS. 6 and 9 show the belt portion 11 subject to a stretching force F_{D} demonstrating the relationship between belt portion 11 extension and the ultimate fit of the article 20. Because the transition from the first appearance state to the second appearance state and ultimately the third appearance state is along a spectrum rather than discrete, in this example, the fit indicator graphic should appear most symmetrical at a total belt portion 11 circumference close to 435 mm. As the belt portion 11 of the “M” sized pull-on article is strained beyond 435 mm in circumference (such as when worn by a larger wearer or otherwise subject to a design exceeding force such as force F_{E} shown in FIGS. 7 and 9), the fit indicator graphic 120 should demonstrate its third appearance state such as third appearance state 127.

In the example of the “M” sized pull-on article given above, when the fit indicator graphic exhibits its third appearance state (e.g. when the belt portion is strained beyond a circumference of 450 mm), the user may be suggested to transition to a larger product offering such as a “large” or “L” sized article. Such an “L” sized article may also be constructed according to the present invention and comprise a fit indicator graphic. The “L” sized article's fit indicator graphic may have a first appearance sate when the belt portion is strained to less than 450 mm. Likewise, such an “L” sized article may have a design fit range from 450 mm to 480 mm in which the fit indicator graphic will demonstrate a second appearance state. The “L” sized article may further be in an overstretched configuration (compared to its design) at a belt portion circumference greater than 480 mm. In this situation, the “L” sized article's fit indicator graphic should demonstrate a third appearance state as described.

To take the example further, a third pull-on article in yet a larger size such as an “XL” size may also be provided. A recommendation, instruction, or suggestion could be provided to a user to utilize this “XL” size article when the “L” sized article described above shows its fit indicator graphic in the third appearance state when being worn by the intended wearer.

Two or more pull-on articles of the present invention may be offered in combination as an array of products. Such an array may be sold or offered for sale as a line-up of product offerings allowing the purchaser to see the size relationship between the members of the array. The fit indicator graphic on the products in the array may provide confirmation that product selected is appropriately sized for the wearer (in the design fit range) or provide an indication that the user should move to a larger size (if the fit indicator graphic appears in its third appearance state) or to a smaller size (if the fit indicator graphic appears in its first appearance state). Such an array maximizes the likelihood that each use of a pull-on article according to the invention will be on an appropriately sized user allowing for optimal product performance of all articles in the array.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An disposable pull-on absorbent article having a waist opening and a pair of leg openings said article comprising a chassis, said chassis having a front portion, a back portion and a crotch portion therebetween, said article further comprising a belt portion, said belt portion comprising a front belt portion, a back belt portion, and left and right side belt portions, said belt portion being disposed transversely to said chassis wherein said side belt portions extend transversely outward from said chassis, and wherein said belt portion defines said waist opening, each of said front belt portion and back belt portion comprising elastomeric material, wherein said waist opening is elastically stretchable and contractable, wherein the elastomeric materials in the belt portion are configured such that the waist opening is characterized by a positive strain design fit range, said design fit range representing less than the full range over which said waist opening may be stretched;
the article further comprising a fit indicator graphic disposed in said belt portion thereof, wherein when said waist opening of said article is strained within a range from its fully contracted state up to the design fit range, said fit indicator graphic has a first appearance, and wherein when said waist opening of said article is strained within said design fit range, said fit indicator graphic has a second appearance, and wherein when said waist opening of said article is strained beyond said design fit range, said fit indicator graphic has a third appearance; wherein by visually confirming that the fit indicator graphic has said second appearance when said article is being worn by a wearer, the correspondence of the wearer to the design fit range of the article may be verified.

2. The absorbent article of claim 1 further comprising a reference graphic, said reference graphic disposed on said chassis outside of said belt region, wherein when said reference graphic coordinates with said second appearance of said fit indicator graphic.

3. The absorbent article of claim 2 wherein said coordination between said reference graphic and said fit indictor graphic is at least substantial identity of appearance.

4. The absorbent article of claim 2 wherein said coordination between said reference graphic and said fit indicator graphic is complementary appearance.

5. The absorbent article of claim 2 wherein said reference graphic comprises a reference color and wherein said fit indicator graphic matches said reference color when said fit indictor graphic has said second appearance.

6. The absorbent article of claim 5 wherein said fit indicator graphic in said first appearance, said second appearance, and said third appearance vary in color.

7. The absorbent article of claim 5 wherein said fit indicator graphic in said first appearance, said second appearance, and said third appearance, vary in intensity of a substantially similar color.

8. The absorbent article of claim 1 wherein said full range over which said waist opening may be stretched exceeds said design fit range by at least 100%.

9. The absorbent article of claim 8 wherein said full range over which said waist opening may be stretched exceeds said design fit range by at least 200%.

10. An array disposable pull-on articles, said array comprising a first article according to claim 1 and a second article according to claim 1, wherein said design fit range of said first article is smaller than said design fit range of said second article and wherein the range of waist opening strain corresponding to said third appearance of said fit indicator graphic of said first article at least partially overlaps the design fit range of said second article.
